(19) United States
(12) Patent Application Publication
(10) Pub. No.: US 2010/0217150 A1
(43) Pub. Date: Aug. 26, 2010

(54) INTERCAVERNOSAL PRESSURE MEASURING INSTRUMENT AND METHOD

(76) Inventors: Brian Walter, Aliso Viejo, CA (US); Michael Hays, Aliso Viejo, CA (US)

Correspondence Address: CONNORS ASSOCIATES, 1600 DOVE ST, SUITE 220, NEWPORT BEACH, CA, 92660, US

(21) Appl. No.: 12/733,607

(22) PCT Filed: Sep. 12, 2008
(86) PCT No.: PCT/US2008/010698

§ 371 Date: Mar. 10, 2010

Related U.S. Application Data
(60) Provisional application No. 61/127,815, filed on May 15, 2008.
(60) Provisional application No. 60/993,704, filed on Sep. 14, 2007.

(51) Int. Cl. A61B 5/00

(52) U.S. Cl. 600/561

(57) ABSTRACT

A user measures the intercavernosal pressure of a penis using an instrument held in one hand and axially pressing the instrument against a glans of a penis for a predetermined period. The instrument provides a visual readout indicating the intercavernosal pressure of the penis at the end of this period and includes an exterior display to indicate the measured intercavernosal pressure of the tested penis. The instrument and glans of the penis are disengaged when the readout is provided by the instrument. A control circuit for the display includes a pressure-measuring device within an interior of the instrument that interacts with an internal portion of a contact member upon the internal portion contacting the pressure-measuring device upon axially pressing an external portion of the contact member against the glans of the penis being tested.

### RELATED PATENT APPLICATIONS & INCORPORATION BY REFERENCE

This application is a PCT application filed pursuant to 35 USC 363 and claims priority based on U. S. Provisional Patent Application No. 60/993,704, entitled ““EQM” Erectile quality monitor, intercavernosal pressure device, personal axial rigidometer,” filed Sep. 14, 2007, and U. S. Provisional Patent Application No. 61/127,815, entitled “Intercavernosal pressure measuring instrument and method,” filed May 15, 2008. These related provisional patent applications are incorporated herein by reference and made a part of this application. If any conflict arises between the disclosure of the invention in this PCT application and that in the related provisional patent applications, the disclosure in this PCT application shall govern. Moreover, any and all U.S. patents, U.S. patent applications, and other documents, hard copy or electronic, cited or referred to in this application are incorporated herein by reference and made a part of this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

### BACKGROUND

With the aging increase of the male population as the baby boomers enter their sixties, erectile dysfunction is a growing problem. In order to better evaluate and treat erectile dysfunction, medical doctors and other health care workers and patients need to measure the intercavernosal pressure of a patient's penis. This is an indicator of the rigidity of an erect penis, and its corresponding functionality and health. One device sold by UROAN of Baleares, Spain, is currently in use. It is inconvenient to simultaneously hold, operate, and read an off center displayed on its front side. It is also costly because its uses an expensive micro-switch to measure the rigidity of the penis.

### SUMMARY

Our instrument for, and method of, measuring the intercavernosal pressure of a penis has one or more of the features depicted in the embodiment discussed in the section entitled “DETAILED DESCRIPTION OF ONE ILLUSTRATIVE EMBODIMENT.” The claims that follow define our instrument for, and method of, measuring the intercavernosal pressure of a penis, distinguishing them from the prior art; however, without limiting the scope of our instrument and method as expressed by these claims in general terms, one or more, but not necessarily all, of their features are:

One, our instrument for measuring intercavernosal pressure of a penis includes a housing sized and configured to be grasped in one hand of a user during measurement. For example, the housing may have a length of approximately 4.5 inches, a width from approximately 2.25 to approximately 2.5 inches with its proximal and distal ends approximately the same dimension of approximately 2.25 and its maximum dimension of approximately 2.5 centrally located between its proximal and distal ends, and a width of approximately 1.25 inch. The housing has a longitudinal axis and opposed longitudinal sides. Each longitudinal side may include a pair of adjacent indentations each sized to receive an individual finger or thumb of a user. These longitudinal sides with the indentations may be symmetrical. The housing may have a substantially oval cross-section. This ergonomic configuration of the housing provides a comfortable “feel” and enhances its ease of use.

Two, a contact member mounted to an end of the housing moves inwardly into an interior of the housing. The contact member may have an external portion extending outwardly from this end of the housing and an internal portion extending inwardly into the interior of the housing. The external portion may terminate in an oval contact body element having a concave outer surface. The oval cross-section of the housing may have dimensions greater than dimensions of the oval contact body's dimensions. The housing and the oval contact body may be concentric about the longitudinal axis with their respective major and minor axes being in registration.

Three, there is a control circuit for an exterior display that indicates the intercavernosal pressure of a penis. The control circuit includes a pressure-measuring device within the interior of the housing that interacts with the internal portion of the contact member upon the internal portion contacting the pressure-measuring device upon axially pressing the external portion of the contact member against a tip of the glans of a penis. The pressure-measuring device may be a transducer. The circuit may include a power on and off switch mounted at or near a proximal end of the instrument and between the display and the contact member and a battery low light mounted at or near a proximal end of the instrument. The power on and off switch may be within a detachable end cap member of the housing. The end cap member may include a moveably mounted transparent or translucent actuator element. The actuator element may be aligned with a light element that is illuminated upon depressing the actuator element.

Four, the display may have a substantially linear configuration and may comprise a plurality of lights in a row longitudinally oriented and centrally located on a front side of the housing. These lights may be discrete light elements that are longitudinally oriented and substantially parallel to the longitudinal axis. Each individual light element may correspond to a different intercavernosal pressure of a penis. There may be indicia nearby each light element indicating a different intercavernosal pressure for each light element. The indicia may be a number or numbers indicating a pressure or pressure ranges, with the indicia numbers proceeding from a lower to a higher number. The control circuit may include a timer element that after a predetermined period turns on the light element that corresponds to the intercavernosal pressure of the penis being measured. The light elements may be color-coded with some light elements when illuminated showing that the intercavernosal pressure of the penis is satisfactory and other light elements when illuminated showing that the intercavernosal pressure of the penis is unsatisfactory.

In one embodiment of our instrument the housing has a front with a plurality of centrally located, longitudinally oriented windows therein, and a plurality of light emitting diodes are mounted on a first circuit board contained in an interior of the housing. Each individual diode is adjacent an individual window so light from an energized diode shines through an adjacent window. Each diode corresponds to a different intercavernosal pressure, or pressure range, of a penis being tested. At least some of the diodes may have different colors. The front has on an external surface thereof indicia nearby each window indicating intercavernosal pressure, or pressure range, for the penis being tested. A contact member is mounted to a proximal end of the housing to move inwardly into the interior of the housing a distance not to exceed approximately ⅛ inch. This contact member has an external portion extending outwardly from the proximal end of the housing and an internal portion extending inwardly into the interior of the housing. The external portion has an exterior concave surface for contacting the tip of the glans of the penis being tested. A power control switch is mounted to a second circuit board contained in the interior of the housing nearby the proximal end. This second circuit board is positioned with respect to the internal portion of the contact member to avoid interfering with the movement of the internal portion of the contact member. A control circuit for the display includes a transducer within the interior of the housing that interacts with the internal portion of the contact member upon the internal portion contacting the transducer upon axially pressing the external portion of the contact member against a glans of a penis.

This embodiment of our instrument may have additional features, for example: (1) It may include a power on/off light mounted on the second circuit board opposite a window in the proximal end so that, upon energizing of the power on/off light, the light's radiation shines through the window. (2) There may be a moveably mounted actuator element seated in the window and functions as an actuator element for the power on/off light. (3) It may be powered by one or more batteries contained within the interior of the housing, and it may include a battery low light mounted on the second circuit board and projecting at least partially from a proximal end of the housing.

Our method of measuring the intercavernosal pressure of a penis comprises the steps of

(a) with a hand held instrument being held in one hand of a user, axially pressing the instrument against the tip of a glans of a penis for a predetermined period until the instrument provides a readout at the end of said period indicating the intercavernosal pressure of the penis,

said instrument having a housing with a front side including a longitudinally oriented, linear visual display for the readout, said housing being sized and configured and the display being centrally located so that, while the user grasps the instrument with one hand, the user's forgers or thumb are directed by indentations in opposed sides of the housing so they do not cover the display, enabling the user to see all of the display,

(b) disengaging the instrument and the glans of the penis when the readout is provided by the instrument.

These features are not listed in any rank order nor is this list intended to be exhaustive.

### DESCRIPTION OF THE DRAWING

One embodiment of our instrument for measuring intercavernosal pressure of a penis is discussed in detail in connection with the accompanying drawing, which is for illustrative purposes only. This drawing includes the following figures (FIGS.), with like numerals indicating like parts:

FIG. 1 is a perspective view of one embodiment of our instrument for measuring intercavernosal pressure of a penis.

FIG. 1A is a perspective view of the embodiment shown in FIG. 1 being held in one hand and a proximal end cap of the instrument's housing partially removed.

FIG. 2 is a perspective view of the embodiment shown in FIG. 1 with the back cover and end cap of the instrument's housing removed.

FIG. 3 is an exploded perspective view of the housing of the embodiment of our instrument shown in FIG. 1.

FIG. 3A is a rear plan view, with the back cover removed, of the embodiment of our instrument shown in FIG. 1.

FIG. 3B is a front plan view of the embodiment of our instrument shown in FIG. 1.

FIG. 3C is a bottom plan view of the embodiment of our instrument shown in FIG. 1.

FIG. 4 is a perspective view of our instrument shown in FIG. 1 partially disassembled showing some of its electrical and electronic components.

FIG. 4A is a perspective view of a switch circuit board used in our instrument shown in FIG. 1.

FIG. 5A is a perspective view of the front side of the display circuit board used in our instrument shown in FIG. 1.

FIG. 5B is a perspective view of the display circuit board shown in FIG. 5A inverted to show its backside.

FIG. 6A is a perspective view looking from the front into the interior of an assembly of a proximal end cap and a contact member used in our instrument shown in FIG. 1.

FIG. 6B is a perspective view looking from the back into the interior of the assembly shown in FIG. 6A.

FIG. 7 is a fragmentary, exploded perspective view of our instrument partially disassembled showing the positional relationship of the assembly of the proximal end cap and contact member and a pressure-measuring device used in our instrument shown in FIG. 1.

FIG. 8 is a table setting forth an example of the specifications of the pressure-measuring device.

FIG. 8A is a table setting forth specification for the light elements of the display.

FIGS. 9A and 9B combined comprise a schematic diagram of the control circuit of the embodiment of our instrument shown in FIG. 1.

FIG. 10A is a plan view of the embodiment of our instrument shown in FIG. 3 being used to measure the intercavernosal pressure of a rigid penis.

FIG. 10B is a plan view of the embodiment of our instrument shown in FIG. 3 being used to measure the intercavernosal pressure of a flaccid or semi-flaccid penis.

### DETAILED DESCRIPTION OF ONE ILLUSTRATIVE EMBODIMENT

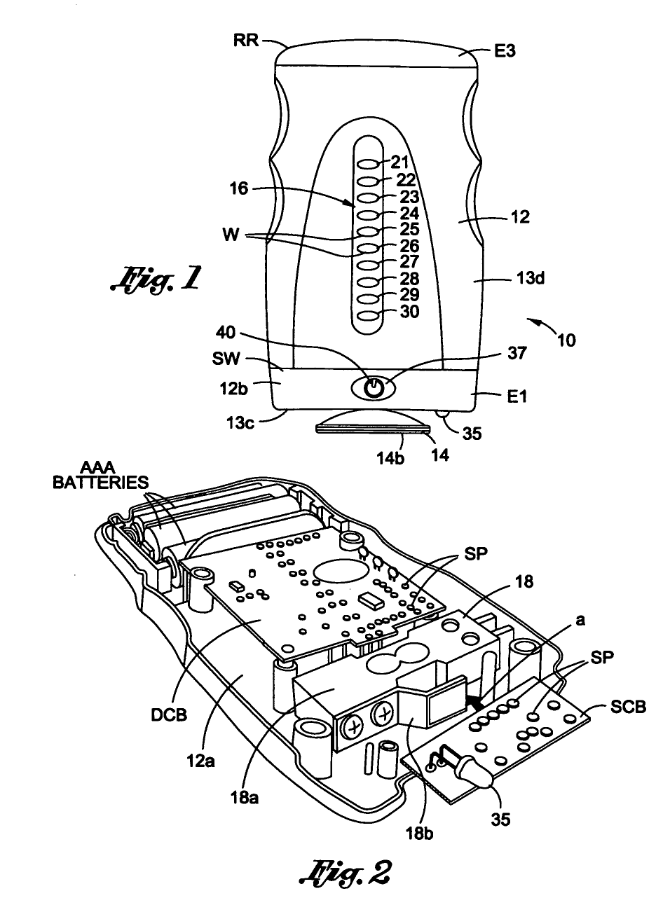

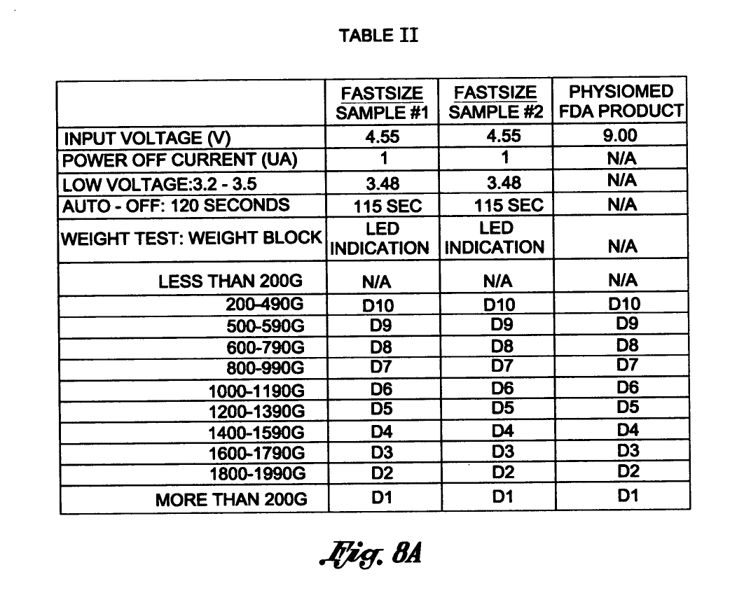

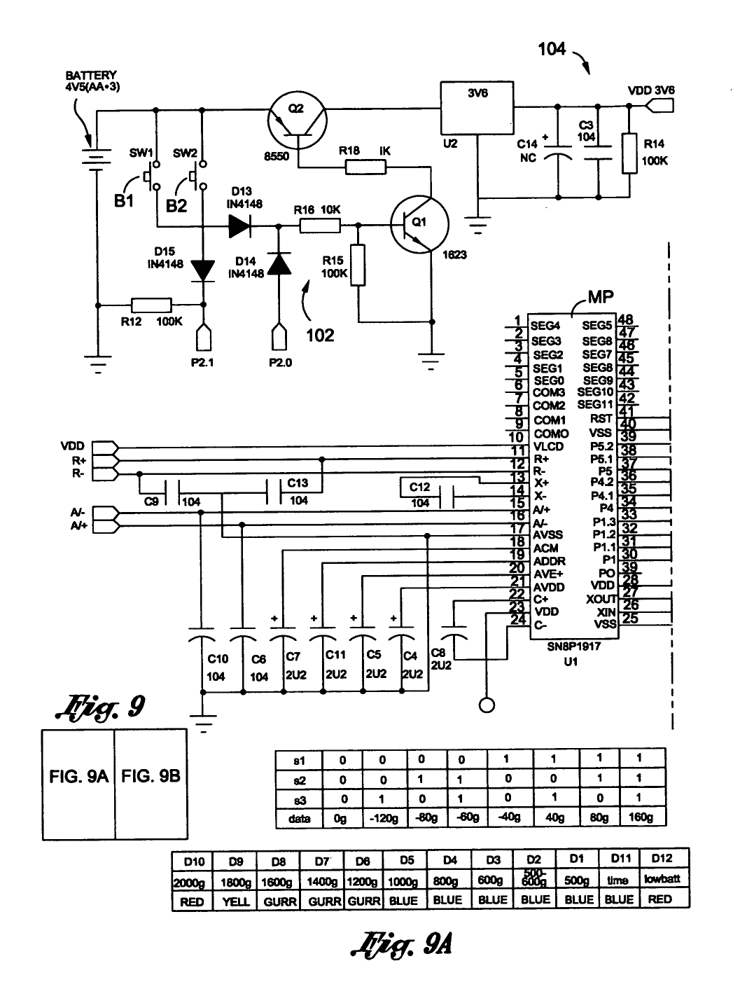

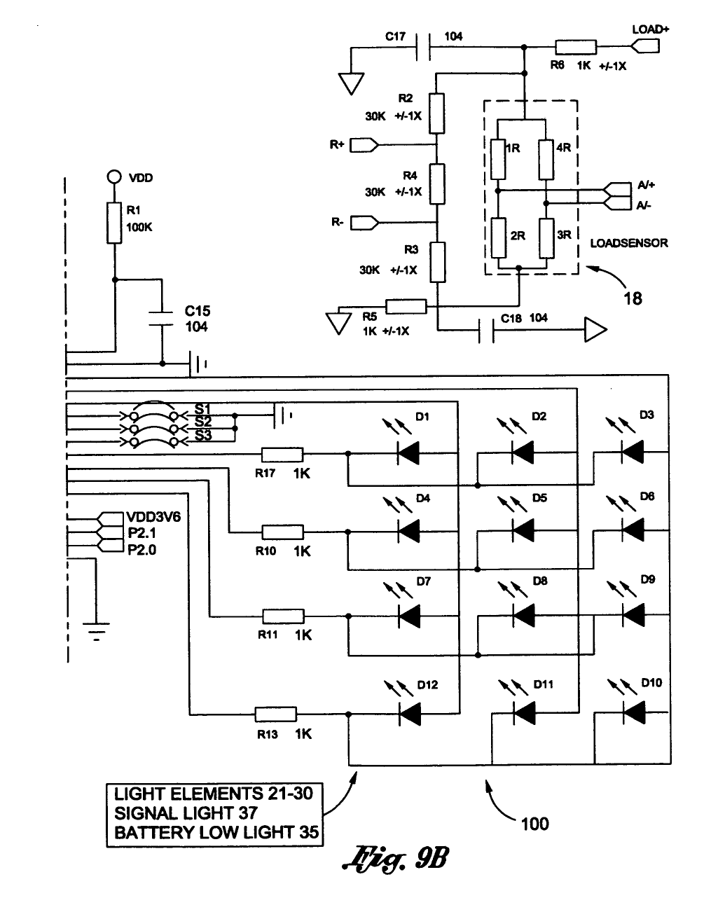

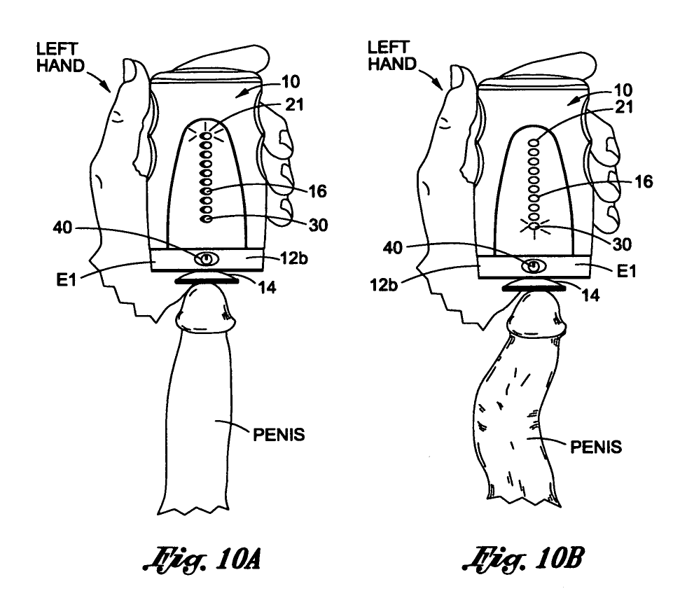

As shown best in FIGS. 1 through 3C, one embodiment our instrument for measuring the intercavernosal pressure of a penis is designated by the numeral 10. It is inexpensive to manufacture and comfortable to hold with one hand and see the readout on its linear, exterior display 16 that indicates the intercavernosal pressure of the penis being tested. A contact member 14 is mounted to a proximal end El of the housing 12. A control circuit 100 (FIG. 9) for our instrument 10 includes a pressure-measuring device 18 within an interior 12a of the housing 12. The pressure-measuring device 18 interacts with an internal portion 14a of the contact member 14 upon this internal portion contacting the pressure-measuring device upon axially pressing an external portion 14b of the contact member against a glans of a penis as shown in FIGS. 10A and 10B. The pressure-measuring device 18 may be a transducer, for example, a load cell or strain gauge. The specifications of this device are set forth in the TABLE I shown in FIG. 8.

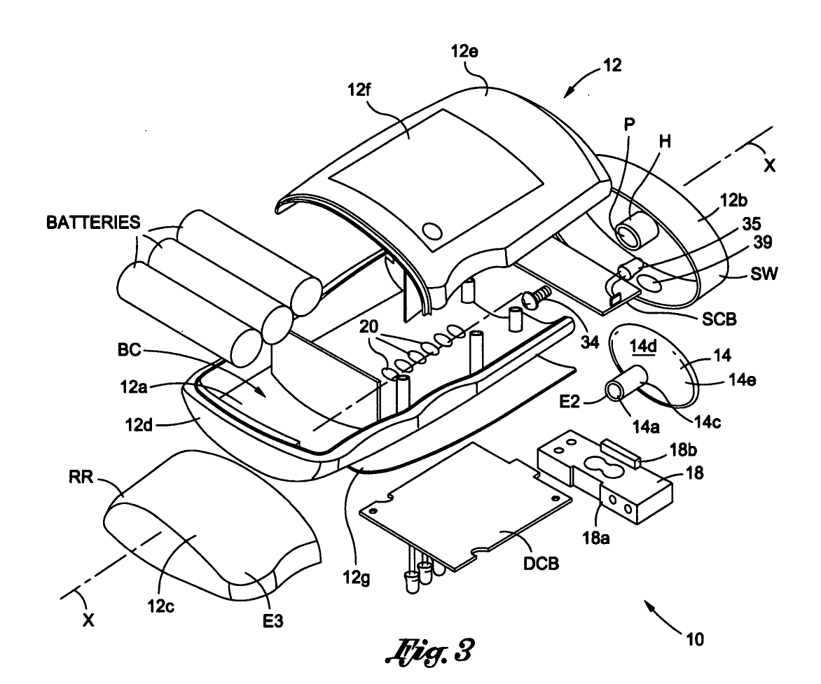

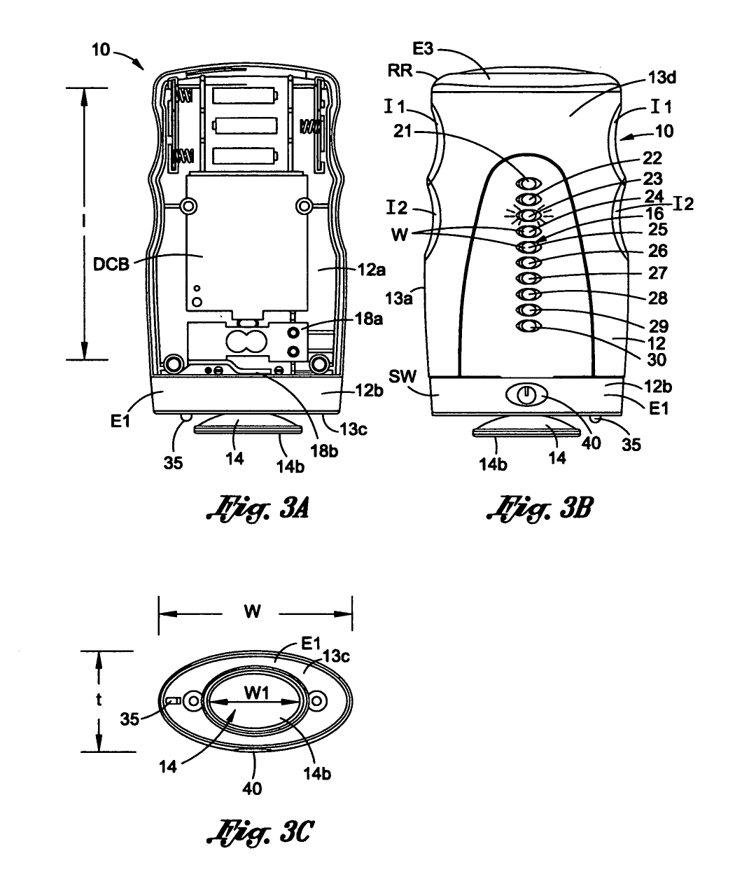

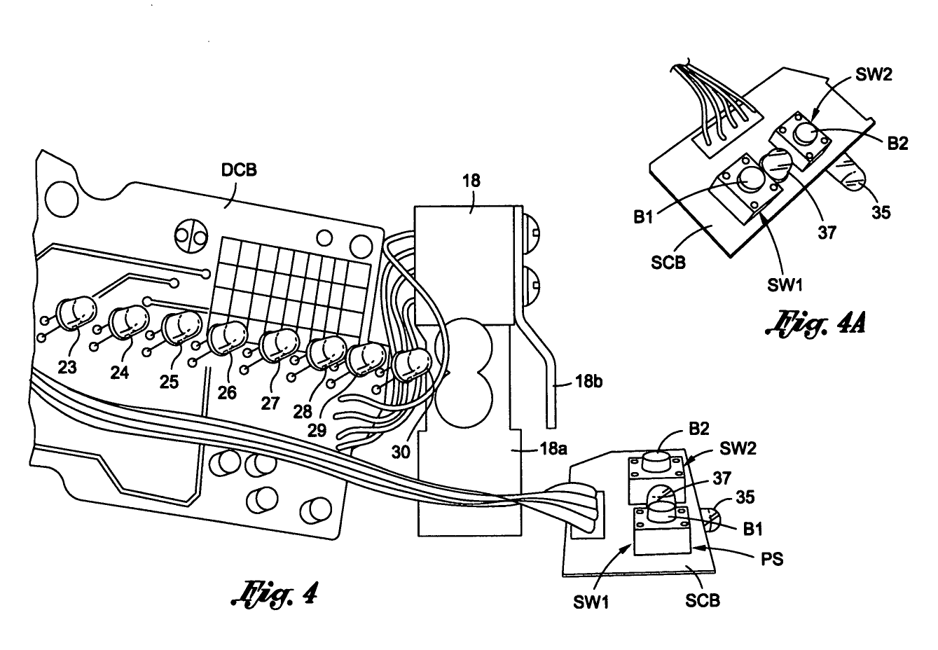

Our instrument 10 is ergonomically superior to the UROAN device. Its housing 12 is sized and configured so the user may hold our instrument 10 with either hand. Those dominantly right-handed normally would hold our instrument 10 in their left hand. Those dominantly left-handed normally would hold our instrument 10 in their right hand. The housing 12 has a longitudinal axis X (FIG. 3) and opposed longitudinal sides 13a and 13b (FIG. 3B). Each longitudinal side 13a and 13b includes a pair of adjacent indentations 11 and 12, each indentation sized to receive an individual finger or thumb, as the case may be, of a user.

The housing 12 has a substantially oval cross-section and its sides 13a and 13b are symmetrical about the axis X. Consequently, each side 13a and 13b has an arc shaped cross-section. The distal end E3 (FIGS. 1, 3, and 3B) of the housing 12 is slightly convex with a rim RR having rounded edges. The housing 12 has: A length I (FIG. 3A) of approximately 4.5 inches. A width w from approximately 2.25 to approximately 2.5 inches with its proximal and distal ends approximately the same width dimension of approximately 2.25 and its maximum dimension of approximately 2.5 centrally located between its proximal and distal ends. And a thickness t (FIG. 3C) of approximately 1.25 inch. As best shown in FIG. 3, the housing 12 comprises a number of detachably connected pieces that may be molded from a plastic material and may be screwed or forced fitted or otherwise assembled to form the housing as an enclosure for the electrical and electronic components of the control circuit 100. The housing pieces may include a proximal end cap 12b, a distal end cap 12c, a front panel 12d, a back panel 12e with a detachable cover 12f, and a display panel 12g. The front panel 12d has aligned holes 20 (FIG. 3) in a row that is substantially parallel to the longitudinal axis X of our instrument 10. The display panel 12g overlies the exterior of the front panel 12d and has open or transparent or translucent windows W (FIGS. 1 and 1A) covering the holes 20. The housing 12 upon connection of its pieces in a conventional manner provides an enclosure for: a battery compartment BC, a display circuit board DCB carrying a plurality of discrete light elements 21 through 30, a switch circuit board SCB, the pressure-measuring device 18, and the internal portion 14a of the contact member 14. The light elements 21 through 30 may, for example, be light emitting diodes (LED's).

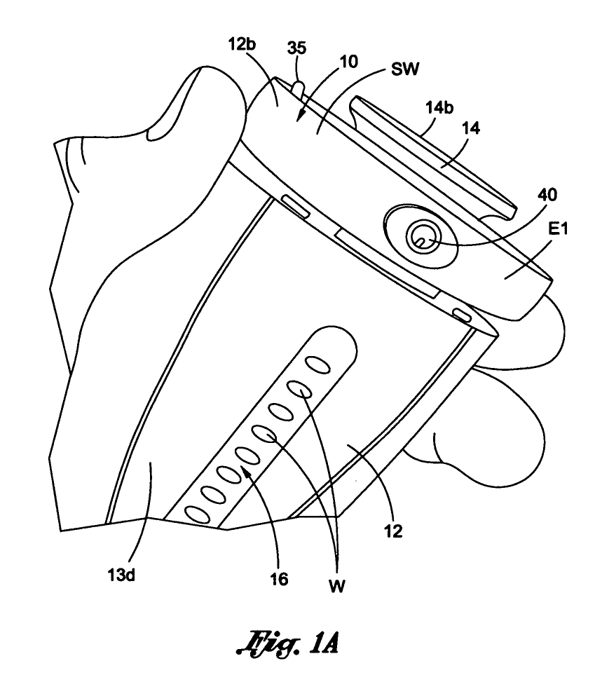

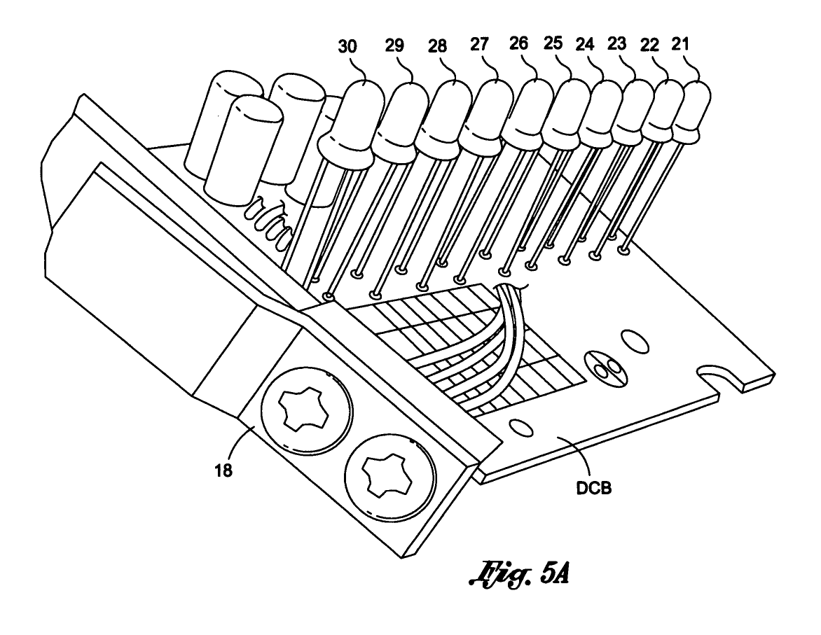

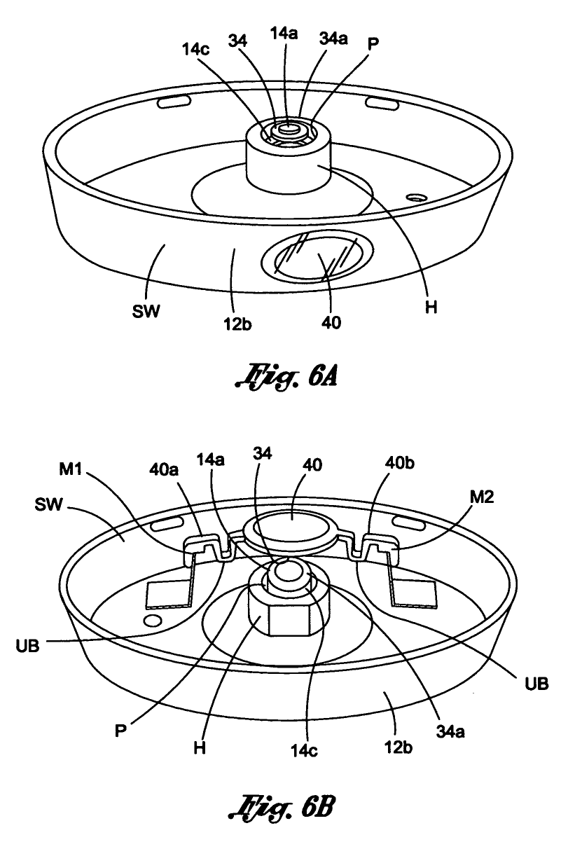

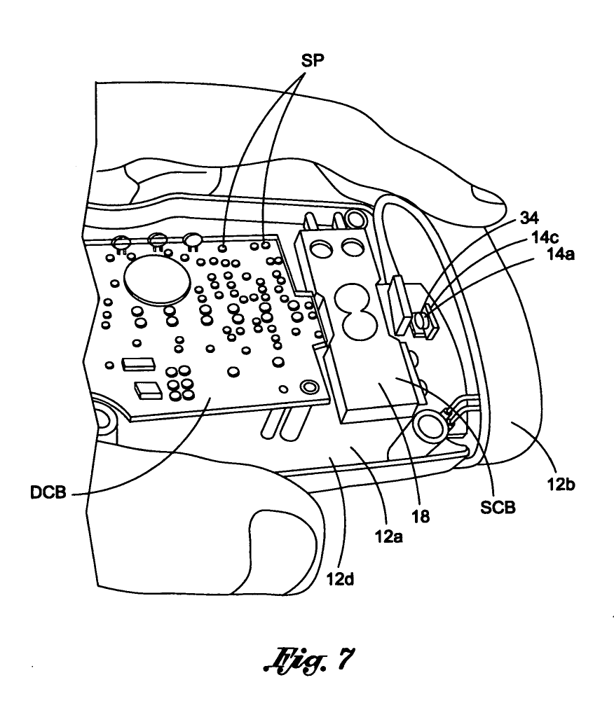

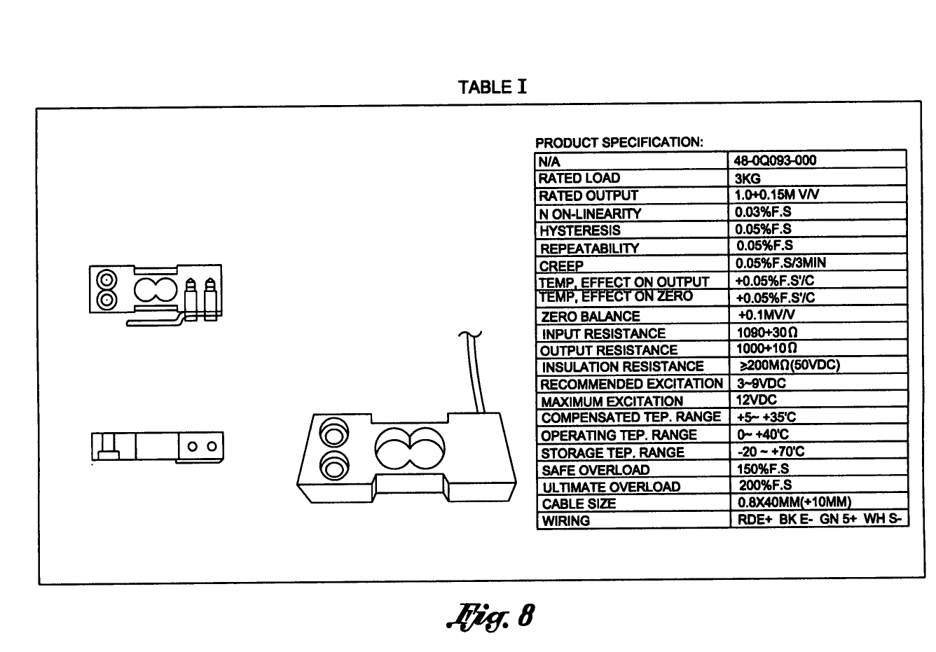

As best illustrated in FIG. 2, the display circuit board DCB is positioned between the battery compartment BC and the pressure-measuring device 18, and the switch circuit board SCB is shown aligned to be inserted beneath the pressure-measuring device 18 as viewed in FIG. 2. This avoids interfering with the movement of the contact member 14. A signal light 37 (FIG. 4A) is mounted on the switch circuit board SCB between of a pair manually actuated mechanical switches SW1 and SW2. The switches SW1 and SW2 respectively have buttons B1 and B2 that are simultaneously depressed to actuate the switches at the same time. A battery low light 35 protruding slightly from the substantially flat bottom side 13c of the housing 12 is an optional feature. These lights 35 and 37 may be LED's. The various electronic components of the control circuit 100 are connected together in a conventional fashion with wires or wire bundles and, where required, they are soldered in place. For example as depicted in FIG. 2, solder points SP on the backsides of the display circuit board DCB and the switch circuit board SCB are exposed when the back panel 12e is removed. Removal of the distal end cap 12c exposes the battery compartment BC that holds three (3) AAA batteries, and detaching cover 12f exposes the display circuit board DCB. As depicted in FIGS. 6A and 6B, the proximal end cap 12b has a sidewall SW with a window opening 39 (FIG. 3) therein covered by a moveable lens 40 made of transparent or translucent material. The lens 40 includes a pair of arms 40a and 40b (FIG. 6B) that are snapped into locking engagement with mounts M1 and M2 integral with the internal surface of the sidewall SW on opposed sides of the lens. Each arm 40a and 40b includes a U-shaped bend. On release of the lens 40, a spring (not shown) returns the lens to its original position offset from the buttons B1 and B2. This manner of mounting holds the lens 40 in position next to the sidewall SW, and covering the window opening 39, but allows the lens to be pushed inward to activate and deactivate the switches SW1 and SW2. Referring to FIGS. 2 and 7, with the switch circuit board SCB positioned within the interior 12a of the housing 12, the side of the switch circuit board carrying the switches SW1 and SW2 faces the lens 40 and is directly beneath it. So depressing the lens 40 inward engages the switches SW1 and SW2 simultaneously as shown in FIGS. 4 and 4A, depressing the buttons B1 and B2 to turn the signal light 37 on or off. Thus, the lens 40 functions as an actuator element for the signal light 37. When this light 37 is energized upon first depressing the lens 40, the light's radiation shines through the lens 40 to indicate that power is on. Depressing the lens 40 again engages the switches SW1 and SW2, shutting off electrical energy. Mounted to the other side of the switch circuit board SCB is the battery low light 35 that the control circuit 100 energizes upon detecting this condition.

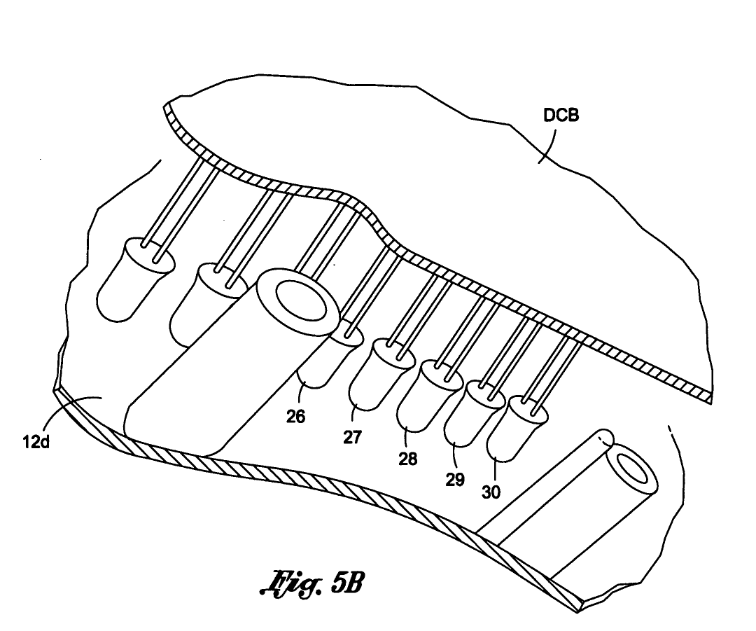

As best shown in FIGS. 1 and 3C, the light elements 21 through 30 are spaced apart in a row that is substantially parallel to the longitudinal axis so they are longitudinally oriented and centrally located on a front side 13d of our instrument's housing 12. Each individual light element 21 through 30 corresponds to a different intercavernosal pressure, or pressure range, of the penis being tested. The control circuit 100 controls the illumination of an individual light element 21 through 30 that corresponds to the intercavernosal pressure of the penis being tested. As best shown in FIGS. 3B and 5A, the light elements 21 through 30 may be color-coded with some light elements when illuminated showing that the intercavernosal pressure of the penis is satisfactory and other light elements when illuminated showing that the intercavernosal pressure of the penis is unsatisfactory. For example, the light elements 21 through 25 are blue corresponding to very satisfactory, the light elements 26 through 28 are green corresponding to acceptable, the light element 29 is yellow corresponding to problematic, and the light element 30 is red corresponding to unsatisfactory. The individual holes 20 in the front panel 12d are in registration with individual ones of the light elements 21 through 30. On the exterior of the display panel 12g nearby each light element 21 through 30 are indicia 32 that indicate a different intercavernosal pressure, or pressure range, for each light element. The indicia 32 may be a number or numbers indicating a pressure or pressure ranges. The indicia numbers proceed from a lower to a higher number sequentially. For example, the light element 21 corresponds to a pressure of +2000 grams, the light element 22 corresponds to a pressure of +1800 grams, the light element 23 corresponds to a pressure of +1600 grams, the light element 24 corresponds to a pressure of +1400 grams, the light element 25 corresponds to a pressure of +1200 grams, the light element 26 corresponds to a pressure of +1000 grams, the light element 27 corresponds to a pressure of +800 grams, the light element 28 corresponds to a pressure of +600 grams, the light element 29 corresponds to a pressure of 500-600 grams, and the light element 30 corresponds to a pressure of less than 500 grams. Each individual indicia number corresponding to an individual light element is directly beneath the corresponding light element. The indicia number may also be above its corresponding light element. In either case the indicia numbers and lights elements 21 through 30 are in the same centrally located, linear, longitudinal row.

As best illustrated in FIGS. 6A and 6B, the contact member 14 is seated on the proximal end cap 12b and is mounted so it may move inwardly when our instrument 10 is used as shown in FIGS. 10A and 10B. Typically this movement does not exceed ⅛ inch, and the normal range of movement in and out relative the to the proximal end cap 12b or the end E1 of the housing 12 is substantially from 1/32 to ⅛ inch. The proximal end cap 12b has a passageway P (FIG. 3) that extends inward through an internal hub H into the interior 12a of the housing 12 along the longitudinal axis X. The contact member 14 includes a stem 14c projecting from a convex side 14d (FIG. 3) of an oval body 14e. The stem 14c is axially oriented substantially along the longitudinal axis X when extending onto the hub H upon assembly of the parts of our instrument. A concave side 14f (FIG. 3C) of the oval body 14e forms the external portion 14b of the contact member 14 that is pressed against a glans of a penis as shown in FIGS. 10A and 10B. The stem 14c is inserted into the passageway P and a screw 34 with an enlarged head 34a is screwed into a threaded receptacle in the end E2 (FIG. 3) of the stem. Because the enlarged head 34a has a diameter greater than the diameter of the passageway P of the hub H, this fastens the contact member 14 to the hub H of the proximal end cap 12b. The screw 34 is loosely tightened to enable movement between the contact member 14 and the proximal end cap 12b. The enlarged head 34a limits outward movement of the contact member 14 relative to the end El and the convex side 14d of the oval body 14e limits inward movement of the contact member. The head 34a of the screw 34 constitutes the internal portion 14a of the contact member 14 that contacts the pressure-measuring device 18 upon inward movement of the contact member and transfers the load or pressure to the pressure-measuring device.

The housing 12 and the oval body 14e are concentric about the longitudinal axis X and their respective major and minor axes are in registration, with the major axes in parallel and extending along the width w of the housing 12 as shown in FIG. 3C, and respectively intersecting the centers of the side 13c of the housing 12 and the center of the oval body 14e. The oval cross-section of the housing 12 has dimensions greater than dimensions of the oval body's dimensions. For best results, the minimum width w1 (FIG. 3C) of the oval body 14e is 1.25 inch. Its height may be less than one (1) inch. These dimensions of the oval body 14e and oval body 14e having its concave side 14f in contact with the tip of the glans during testing, minimizes or avoids sideway slippage of the tip of the glans during testing.

The pressure-measuring device 18 is a conventional device. It includes a block 18a and a bent arm 18b attached to the block. In a rest condition the enlarged head 34a of the screw 34 of the contact member 14 is offset from the bent arm 18b. In the measure condition the concave side 14f of the oval body 14e (the external portion 14b of the contact member 14) presses against the tip a glans of a penis as shown in FIGS. 10A and 10B. The pressure that the penis exerts against the concave side 14f is transferred to the bent arm 18b upon the head 34a of the screw 34 bearing against the bent arm. When this occurs, the pressure-measuring device 18 generates an electrical signal corresponding to the detected pressure. This electrical signal through the control circuit 100 activates the individual light element of the light elements 21 through 30 that corresponds to the detected pressure. The user holds our instrument 10 firmly against the tip of the glans of the penis as shown in FIGS. 10A and 10B until this one light element is illuminated after a predetermined time period elapses. For example as shown on FIG. 3B, the light element 23 is illuminated to indicated that the penis pressure was +1600 grams. The control circuit 100 includes a timer function that, after expiration of the predetermined period, turns on the light element that corresponds to the intercavernosal pressure of the penis being tested.

### Control Circuit

As illustrated in FIG. 9, the control circuit 100 includes a conventional microprocessor MP programmed in the conventional manner. A suitable microprocessor MP is sold by Sonix Technology Co., Lt, under the designation SN8P1917. This microprocessor MP operates which of the light elements 21 through 30 is illuminated when the contact member 14 transfers the pressure exerted against it by the penis to the arm 18b of the pressure-measuring device 18. The pressure-measuring device 18 converts this pressure into an electrical signal that energizes one of the light elements 21-30, as the case may be, corresponding to this pressure. When a user depresses the lens 40 to actuate the switches SW1 an SW2, which are normally in an off position, a transistor switch circuit 102 is energized to apply power under the control of a voltage regulator circuit 104 to the circuit 100. Electrical energy is provided to the signal light 37 to illuminate it, alerting the user that the instrument is ready to take a measurement. As the tip of the glans of the penis presses against the concave side 14f (FIG. 3C) of the oval body 14e, advancing the contact member 14 inwardly, the arm 18b of the pressure-measuring device 18 is deflected inwardly slightly in response to the pressure. As soon as contact is made, the microprocessor MP turns on the signal light 37. After a predetermined time period of from approximately 1 to 5 approximately seconds, the signal light 37 blinks off and then on and the readout of the measured rigidity is provided by one of the light elements 21-30 being illuminated. The signal light 37 will then remain constantly illuminated until the lens 40 is again depressed to actuate the switches SW1 and SW2 to disconnect the AAA batteries and discontinue supplying energy to the circuit 100.

### Method of Use

As illustrated in FIGS. 10A and 10B, our method of measuring intercavernosal pressure of a penis comprises axially pressing the external portion 14b of the contact member 14 against the tip of the glans of the penis for no more than, for example, approximately 5 seconds. The user only needs one hand to hold the instrument 10 while performing the measurement. As discussed above, our instrument 10 provides a visual readout indicating the intercavernosal pressure of the penis at the end of this brief time period. The user relieves the pressure by disengaging contact between the instrument 10 and the glans of the penis when the instrument provides the readout. FIG. 10A depicts a very rigid penis being tested showing the blue light element 21 illuminate to show a readout corresponding to very satisfactory. FIG. 10B depicts a flaccid penis being testing showing the red light element 30 illuminate to display a readout corresponding to an unsatisfactory erection.

Because the visual display 16 is longitudinally oriented, linear, and centrally located on the front side 13d, and because the housing 12 is sized and configured as discussed above, while the user grasps the instrument with one hand, the user's fingers and thumb of this hand are directed by indentations I1 and I2 to avoid covering the display. Since the fingers and thumb do not cover the display 16, the user is able to see all of the display without the user's hand blocking or interfering with seeing the readout.

### SCOPE OF THE INVENTION

The above presents a description of the best mode we contemplate of carrying out our instrument, and of the manner and process of making and using it, in such full, clear, concise, and exact terms as to enable any person skilled in the art to which it pertains to make and use our instrument. Our instrument is, however, susceptible to modifications and alternate constructions from the illustrative embodiment discussed above which are fully equivalent. Consequently, it is not the intention to limit our instrument to the particular embodiment disclosed. On the contrary, our intention is to cover all modifications and alternate constructions coming within the spirit and scope of our instrument as generally expressed by the following claims, which particularly point out and distinctly claim the subject matter of our invention:

1. An instrument for measuring intercavernosal pressure of a penis comprising
a housing sized to be grasped in the hand of a user, said housing having a front with a plurality of centrally located, longitudinally oriented windows therein,
a plurality of light emitting diodes mounted on a first circuit board contained in an interior of the housing, each individual diode being adjacent an individual window so light from an energized diode shines through an adjacent window, each diode corresponding to a different intercavernosal pressure, or pressure range, of a penis being tested, at least some of the diodes having different colors,
said front having on an external surface thereof indicia nearby each window indicating intercavernosal pressure, or pressure range, for the penis being tested,
a contact member mounted to a proximal end of the housing to move inwardly into the interior of the housing a distance not to exceed approximately ⅛ inch,
said contact member having an external portion extending outwardly from said proximal end of the housing and an internal portion extending inwardly into said interior of the housing,
said external portion having an exterior concave surface for contacting the tip of the glans of the penis being tested,
a power control switch mounted to a second circuit board contained in the interior of the housing nearby said proximal end, said second circuit board being positioned with respect to the internal portion of the contact member to avoid interfering with the movement of said internal portion of the contact member, and
a control circuit for the display including a transducer within said interior of the housing that interacts with the internal portion of the contact member upon said internal portion contacting the transducer upon axially pressing the external portion of the contact member against a glans of a penis.

2. The instrument of claim 1 where a power on/off light is mounted on the second circuit board opposite a window in the proximal end so that, upon energizing of the power on/off light, the light's radiation shines through the window.

3. The instrument of claim 2 where a moveably mounted actuator element is seated in the window and functions as an actuator element for the power on/off light.

4. The instrument of claim 1 where it is powered by one or more batteries contained within the interior of the housing, and the instrument includes a battery low light mounted on the second circuit board and projecting at least partially from a proximal end of the housing.

5. An instrument for measuring intercavernosal pressure of a penis comprising
a housing sized and configured to be grasped in one hand of a user during measurement, said housing having a longitudinal axis and opposed longitudinal sides, each longitudinal side including a pair of adjacent indentations each sized to receive an individual finger or thumb of a user, said sides being substantially symmetrical,
a housing having a substantially oval cross-section, and a linear, exterior display to indicate the intercavernosal pressure of a penis, said display comprising a plurality of lights in a row longitudinally oriented and centrally located on a front side of the housing,
a contact member mounted to an end of the housing to move inwardly into an interior of the housing,
said contact member having an external portion extending outwardly from said end of the housing and an internal portion extending inwardly into said interior of the housing, said external portion terminating in an oval contact body element having an concave outer surface, the oval cross-section of the housing having dimensions greater than dimensions of the oval contact body's dimensions,
the housing and the oval contact body being concentric about the longitudinal axis with their respective major and minor axes being in registration, and
a control circuit for the display including a pressure-measuring device within said interior of the housing that interacts with the internal portion of the contact member upon said internal portion contacting the pressure-measuring device upon axially pressing the external portion of the contact member against a tip of the glans of a penis.

6. The instrument of claim 5 where the pressure-measuring device is a transducer.

7. The instrument of claim 5 where the housing has a length of approximately 4.5 inches, a width from approximately 2.25 to approximately 2.5 inches with its proximal and distal ends approximately the same dimension of approximately 2.25 and its maximum dimension of approximately 2.5 centrally located between its proximal and distal ends, and a width of approximately 1.25 inch.

8. The instrument of claim 5 including a power on and off switch mounted at or near a proximal end of the instrument and between the display lights and the contact member and a battery low light mounted at or near a proximal end of the instrument.

9. The instrument of claim 8 where the power on and off switch is within a detachable end cap member of the housing and the end cap member includes a moveably mounted transparent or translucent actuator element, said actuator element aligned with a light element that is illuminated upon depressing the actuator element.

10. An instrument for measuring intercavernosal pressure of a penis comprising
a housing sized to be grasped in the hand of a user and including a longitudinal axis, said housing having on an exterior thereof a display comprising a plurality of discrete light elements longitudinally oriented and substantially parallel to the longitudinal axis and centrally positioned on the exterior, each individual light element corresponding to a different intercavernosal pressure of a penis and indicia nearby each light indicating a different intercavernosal pressure for each light element,
a contact member at an end of the housing having an external portion and an internal portion, said contact member axially oriented substantially along the longitudinal axis, and
a control circuit for controlling the light elements, said control circuit including a pressure-measuring device within said interior of the housing that interacts with the internal portion of the contact member upon said internal portion contacting the pressure-measuring device upon axial pressure being exerted on the plunger element by a penis, said control circuit turning on an individual light element that corresponds to the intercavernosal pressure of the penis.

11. The instrument of claim 10 where the pressure-measuring device is a transducer.

12. The instrument of claim 11 where the light elements are spaced apart in a row that is substantially parallel to the longitudinal axis, said indicia is a number or numbers indicating a pressure or pressure ranges, said indicia numbers proceeding from a lower to a higher number.

13. The instrument of claim 12 where the control circuit includes a timer element that after a predetermined period turns on the light element that corresponds to the intercavernosal pressure of the penis.

14. The instrument of claim 10 where the light elements are color-coded with some light elements when illuminated showing that the intercavernosal pressure of the penis is satisfactory and other light elements when illuminated showing that the intercavernosal pressure of the penis is unsatisfactory.

15. An instrument for measuring intercavernosal pressure of a penis comprising
a housing sized to be grasped in one hand of a user, said housing having on an exterior thereof a centrally located, linear display of discrete longitudinally oriented light elements, each individual light element corresponding to a different intercavernosal pressure of a penis and indicia associated with each light indicating a different intercavernosal pressure for each light element, said indicia comprising a number or numbers indicating a pressure or pressure ranges, said individual indicia numbers proceeding from a lower to a higher number, and
a control circuit for controlling the light elements, said control circuit including a transducer mounted at an end of the housing to be interactive with the glans of a penis pressed axially against said end, said control circuit turning on an individual light element that corresponds to the intercavernosal pressure of the penis.

16. The instrument of claim 15 where the control circuit includes a timer element that after a predetermined period turns on the light element that corresponds to the intercavernosal pressure of the penis.

17. The instrument of claim 16 where the light elements are color-coded with some light elements when illuminated showing that the intercavernosal pressure of the penis is satisfactory and other light elements when illuminated showing that the intercavernosal pressure of the penis is unsatisfactory.

18. A method of measuring intercavernosal pressure of a penis comprising the steps of
(a) with a hand held instrument being held in one hand of a user, axially pressing the instrument against the tip of a glans of a penis for a predetermined period until the instrument provides a readout at the end of said period indicating the intercavernosal pressure of the penis,
said instrument having a housing with a front side including a longitudinally oriented, linear visual display for the readout, said housing being sized and configured and the display being centrally located so that, while the user grasps the instrument with one hand, the user's fingers or thumb are directed by indentations in opposed sides of the housing so they do not cover the display, enabling the user to see all of the display,
(b) disengaging the instrument and the glans of the penis when the readout is provided by the instrument.

19. The method of claim 18 where the instrument comprises
a contact member mounted to an end of the housing to move inwardly into an interior of the housing,
said contact member having an external portion extending outwardly from said end of the housing and an internal portion extending inwardly into said interior of the housing, and
a control circuit for the display including a pressure-measuring device within said interior of the housing that interacts with the internal portion of the contact member upon said internal portion contacting the pressure-measuring device upon axially pressing the external portion of the plunger element against a glans of a penis.

20. The method of claim 19 where the pressure-measuring device is a transducer.
